(19) United States
(12) Patent Application Publication
(10) Pub. No.: US 2006/0160810 A1
(43) Pub. Date: Jul. 20, 2006

(54) 2-Alkoxyphenyl substituted imidazotriazinones

(76) Inventors: Ulrich Niewohner, Wermelskirchen (DE); Maria Theresia Niewohner, Wermelskirchen (DE); Helmut Haning, Wuppertal (DE); Martin Radtke, Erkarth (DE); Dietrich Seidel, Wuppertal (DE); Erwin Bischoff, Wuppertal (DE); Wolfgang Karl, Odenthal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/467,966

(22) PCT Filed: Feb. 4, 2002
(86) PCT No.: PCT/EP02/01100

§ 371 Date: Dec. 12, 2003

(30) Foreign Application Priority Data
Feb. 15, 2001 (DE) 101 07 639.8

(51) Int. Cl. A61K 31/53; C07D 487/04

(52) U.S. Cl. 514/243; 544/184

(57) ABSTRACT

The 2-phenyl-substituted imidazotriazinones having short unbranched alkyl radicals in the 9-position are prepared from the corresponding 2-phenyl-imidazotriazinones by chlorosulphonation and subsequent reaction with the amines. The compounds inhibit cGMP-metabolising phosphodiesterases and are suitable for use as active compounds in pharmaceuticals, for treating cardiovascular and cerebrovascular disorders and/or disorders of the urogenital system, in particular for treating erectile dysfunction.

The present invention relates to 2-alkoxyphenyl-substituted imidazotriazinones, to a process for their preparation and to their use as pharmaceuticals, in particular as inhibitors of cGMP-metabolising phosphodiesterases.

The published specification DE 28 11 780 describes imidazotriazines as bronchodilators having spasmolytic activity and inhibitory activity against phosphodiesterases which metabolise cyclic adenosine monophosphate (cAMP-PDEs, nomenclature according to Beavo: PDE-III and PDE-IV). An inhibitory action against phosphodiesterases which metabolise cyclic guanosine monophosphate (cGMP-PDEs, nomenclature according to Beavo and Reifsnyder (Trends in Pharmacol. Sci. 11, 150-155, 1990): PDE-I, PDE-II and PDE-V) has not been described. Compounds having a sulphonamide group in the aryl radical in the 2-position are not claimed. Furthermore, FR 22 13 058, CH 59 46 71, DE 22 55 172, DE 23 64 076 and EP 000 9384 describe imidazotriazinones which do not have a substituted aryl radical in the 2-position and are likewise said to be bronchodilators having cAMP-PDE inhibitory action.

WO 94/28902 describes pyrazolopyrimidinones Which are suitable for treating impotence.

WO 99/24433 and WO 99/67244 describe imidazotriazinones which are suitable for treating impotence.

At this stage, 11 phosphodiesterases having varying specificity for the cyclic nucleotides cAMP and cGMP have been described in the literature (cf. Fawcett et al., Proc. Nat. Acad. Sci. 97(7), 3072-3077 (2000)). Phosphodiesterases which metabolise cyclic guanosine 3′,5′-monophosphate (cGMP-PDE's) are PDE-1, PDE-2, PDE-5, PDE-6, PDE-9, PDE-10 and PDE-11. The compounds according to the invention are potent inhibitors of phosphodiesterase 5. Owing to the different expression of the phosphodiesterases in different cells, tissues and organs, and the differentiated subcellular localization of these enzymes, it is possible, using the selective inhibitors according to the invention, to selectively increase the cGMP concentration in specific cells, tissues and organs, thus addressing different cGMP-regulated processes. This is to be expected in particular in cases where, under certain physiological conditions, the synthesis of cGMP is increased. For example, during sexual stimulation, nitrogen monoxide is released neuronally in the vessels of the Corpus cavemosum, and the synthesis of cGMP is thus increased. This causes a considerable expansion of the vessels which supply the Corpus cavemosum with blood, thus resulting in an erection. Accordingly, inhibitors of cGMP-metabolising PDEs should be particularly suitable for treating erectile dysfunction.

An increase of the cGMP concentration can lead to beneficial antiaggregatory, antithrombotic, antiprolific, antivasospastic, vasodilative, natriuretic and diuretic effects and influence conduction in the central nervous system and thus memory performance. It can influence the short- or long-term modulation of vascular and cardiac inotropism, of pulse and of cardiac conduction (J. C. Stoclet, T. Keravis, N. Komas and C. Lugnier, Exp. Opin. Invest. Drugs (1995), 4 (11), 1081-1100).

The present invention relates to compounds of the general formula (I) 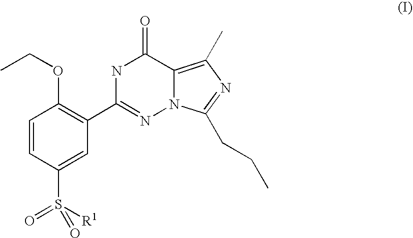
in which
R¹ represents 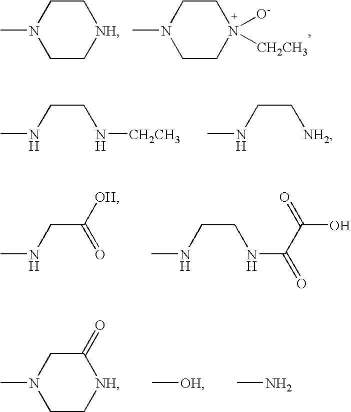
and to their salts and hydrates.

In the context of the invention, preference is given to physiologically acceptable salts. Physiologically acceptable salts can be salts of the compounds according to the invention with inorganic or organic acids. Preference is given to salts with inorganic acids such as, for example, hydrochloric acid, hydrobromic acid, phosphoric acid or sulphuric acid or salts with organic carboxylic or sulphonic acids such as, for example, acetic acid, maleic acid, fumaric acid, malic acid, citric acid, tartaric acid, lactic acid, benzoic acid, or methanesulphonic acid, ethanesulphonic acid, phenylsulphonic acid, toluenesulphonic acid or naphthalenedisulphonic acid.

Physiologically acceptable salts can also be metal or ammonium salts of the compounds according to the invention. Particular preference is, for example, given to sodium, potassium, magnesium or calcium salts, and also to ammonium salts derived from ammonia or organic amines, such as, for example, ethylamine, di- or triethylamine, di- or triethanolamine, dicyclohexylamine, dimethylaminoethanol, arginine, lysine, ethylenediamine or 2-phenylethylamine.

The compounds according to the invention, in particular the salts, can also be present as hydrates. In the context of the invention, hydrates are to be understood as meaning compounds which contain water in the crystal. Such compounds can contain one or more, typically 1 to 5, equivalents of water. Hydrates can be prepared, for example, by crystallizing the compound in question from water or a water-containing solvent.

The compounds according to the invention can be prepared by converting compounds of the formula (II) 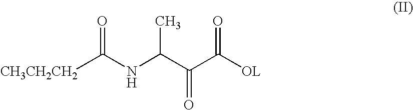
in which
L represents straight-chain or branched alkyl having up to 4 carbon atoms
using the compound of the formula (III) 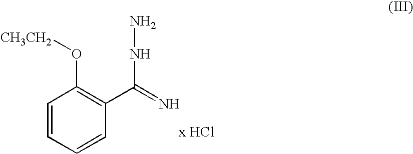
in a two-step reaction in the systems ethanol and phosphorus oxytrichloride/dichloroethane into the compound of the formula (IV) 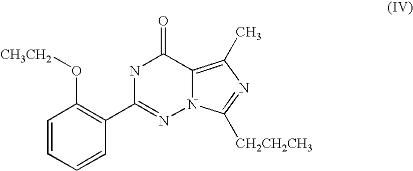
which, in a further step, is converted with chlorosulphonic acid into the compound of the formula (V) 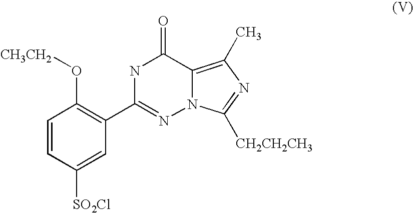
which is subsequently reacted with the corresponding amines in inert solvents to give the sulphonamides or convert it into the free sulphonic acid.

The process according to the invention can be illustrated in an exemplary manner by the equation below:

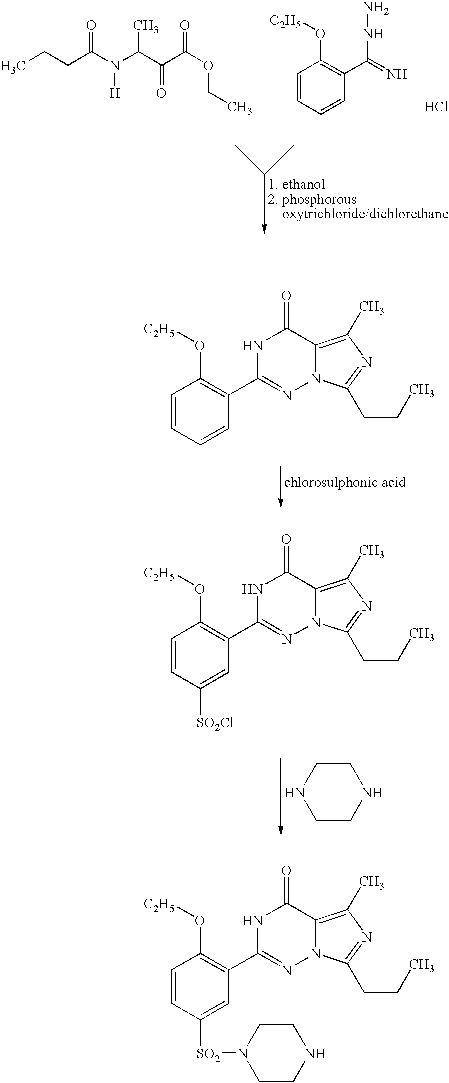

Suitable solvents for the individual steps are the customary organic solvents which do not change under the reaction conditions. These preferably include ethers, such as diethyl ether, dioxane, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethane, trichloroethylene or chlorobenzene, or ethyl acetate, dimethylformamide, hexamethylphosphoric triamide, acetonitrile, acetone, dimethoxyethane or pyridine. It is also possible to use mixtures of the solvents mentioned. Particularly preferably, ethanol is used for the first step and dichloroethane for the second step.

The reaction temperature can generally be varied within a relatively wide range. In general, the reaction is carried out in a range of from −20° C. to 200° C., preferably from 0° C. to 70° C.

The process steps according to the invention are generally carried out at atmospheric pressure. However, it is also possible to operate under elevated or reduced pressure (for example in the range of from 0.5 to 5 bar).

The conversion into the compounds of the formula (V) is carried out in a temperature range of from 0° C. to room temperature and at atmospheric pressure.

The reaction with the corresponding amines is carried out in one of the abovementioned chlorinated hydrocarbons, preferably in dichloromethane.

The reaction temperature can generally be varied within a relatively wide range. In general, the reaction is carried out in a range of from −20° C. to 200° C., preferably from 0° C. to room temperature.

The reaction is generally carried out at atmospheric pressure. However, it is also possible to operate under elevated or reduced pressure (for example in the range of from 0.5 to 5 bar).

The compounds of the formula (II) can be prepared by converting compounds of the general formula (VII)
CH₃CH₂CH₂—CO-T (VII)
in which
T represents halogen, preferably chlorine,
initially by reaction with D, L-alanine of the formula (VIII) 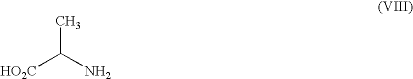
in inert solvents, if appropriate in the presence of a base and trimethylsilyl chloride, into the compound of the formula (IX) 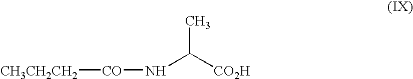
followed by reaction with the compound of the formula (X) 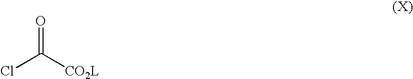
in which
L is as defined above
in inert solvents, if appropriate in the presence of a base.

Suitable solvents for the individual steps of the process are the customary organic solvents which do not change under the reaction conditions. These preferably include ethers, such as diethyl ether, dioxane, tetrahydrofuran, glycol dimethyl ether, or hydrocarbons, such as benzene, toluene, xylene, hexane, cylohexane or mineral oil fractions, or halogenated hydrocarbons, such as dichloromethane, trichloromethane, carbon tetrachloride, dichloroethylene, trichloroethylene or chlorobenzene, or ethyl acetate, dimethylformamide, hexamethylphosphoric triamide, acetonitrile, acetone, dimethoxyethane or pyridine. It is also possible to use mixtures of the solvents mentioned. Particularly preferably, dichloromethane is used for the first step and a mixture of tetrahydrofuran and pyridine for the second step.

Suitable bases are, in general, alkali metal hydrides or alkoxides, such as, for example, sodium hydride or potassium tert-butoxide, or cyclic amines, such as, for example, piperidine, pyridine, dimethylaminopyridine, or C₁-C₄-alkylamines, such as, for example, triethylamine. Preference is given to triethylamine, pyridine and/or dimethylaminopyridine.

The base is generally employed in an amount of from 1 mol to 4 mol, preferably from 1.2 mol to 3 mol, in each case based on 1 mol of the compound of the formula (X).

The reaction temperature can generally be varied in a relatively wide range. In general, the reaction is carried out in a range of from −20° C. to 20° C., preferably from 0° C. to 100° C.

The compounds of the formulae (VII), (VIII) and (X) are known per se.

The compound of the formula (III) can be prepared by reacting the compound of the formula (XI) 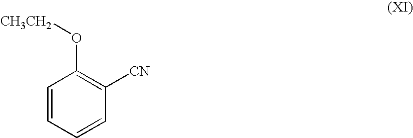
with ammonium chloride in toluene and in the presence of trimethylaluminium in hexane in a temperature range of from −20° C. to room temperature, preferably at 0° C., and at atmospheric pressure and reacting the resulting amidine, if appropriate in situ, with hydrazine hydrate.

The compound of the formula (XI) is known per se.

The compounds according to the invention have an unforeseeable useful pharmacological activity spectrum.

They inhibit cGMP-metabolising phosphodiesterase 5. This results in an increase of cGMP. Owing to the differentiated expression of the phosphodiesterases in different cells, tissues and organs and the differentiated subcellular localization of these enzymes, it is possible, using the selective inhibitors according to the invention, to selectively address the various cGMP-regulated processes.

Moreover, the compounds according to the invention enhance the activity of substances such as, for example, EDRF (endothelium-derived relaxing factor), ANP (atrial natriuretic peptide), of nitrovasodilators and all other substances which increase the cGMP concentration in a manner different from that of phosphodiesterase inhibitors.

The compounds of the general formula (I) according to the invention are therefore suitable for the prophylaxis and/or treatment of disorders where an increase of the cGMP concentration is beneficial, i.e. disorders which are associated with cGMP-regulated processes (in most cases simply referred to as “cGMP-related diseases”). These include cardiovascular disorders, disorders of the urogenital system and also cerebrovascular disorders.

For the purpose of the present invention, the term “cardiovascular disorders” includes disorders such as, for example, hypertension, pulmonary hypertension, stable and unstable angina, peripheral and cardial vasculopathies, arrhythmia, thromboembolic disorders and ischemias such as myocardial infarction, stroke, transitory and ischemic attacks, angina pectoris, obstruction of peripheral circulation, prevention of restinoses after thrombolysis therapy, percutaneous transluminal angioplasty (PTA), percutaneous transluminal coronary angioplasties (PTCA) and bypass.

Furthermore, the compounds of the general formula (I) according to the invention may also be of significance for cerebrovascular disorders. These include, for example, cerebral ischemia, stroke, reperfusion damage, brain trauma, oedema, cerebral thrombosis, dementia, reduced memory performance and Alzheimer's disease.

Owing to their relaxing action on smooth muscles, they are suitable for treating motility disturbances in the digestive tract such as gastroparesis and disorders of the urogenital system such as hypertrophy of the prostate, BHP, incontinence and in particular for treating erectile dysfunction and female sexual dysfunction.

Activity of the phosphodiesterases (PDEs)

To test the inhibiting action, the “Phosphodiesterase [³H] cGMP-SPA enzyme assay” from Amersham Life Science was used. The test was carried out according to the manufacturer's test protocol. Use was made of human recombinant PDE5 which was expressed in a bacculovirus system. The substance concentration at which the reaction rate is reduced by 50% was measured.

### Inhibition of the Phosphodiesterases In Vitro

TABLE 1

| | PDE V |
|---|---|
| Ex. No. | IC₅₀ [nM] |
| 1 | 4.2 |
| 2 | 19 |
| 3 | 19 |
| 4 | 2.4 |

In principle, inhibition of phosphodiesterase 5 results in an increase of the cGMP concentration. Thus, the compounds are of interest for all therapies in which an increase of the cGMP concentration is considered to be beneficial.

The erection-stimulating action was investigated using rabbits which were awake [Naganuma H, Egashira T, Fuji J, Clinical and Experimental Pharmacology and Physiology 20, 177-183 (1993)]. The substances were administered intravenously, orally or parenterally.

The novel active compounds and their physiologically acceptable salts (for example hydrochlorides, maleates or lactates) can be converted in a known manner into the customary formulations, such as tablets, coated tablets, pills, granules, aerosols, syrups, emulsions, suspensions and solutions, using inert non-toxic, pharmaceutically suitable excipients or solvents. In this case the therapeutically active compound should in each case be present in a concentration from approximately 0.5 to 90% by weight of the total mixture, i.e. in amounts which are sufficient to achieve the dosage range indicated.

The formulations are prepared, for example, by extending the active compounds using solvents and/or excipients, if appropriate using emulsifiers and/or dispersants, it optionally being possible, for example, to use organic solvents as auxiliary solvents if the diluent used is water.

Administration is carried out in a customary manner, preferably orally, transdermally or parenterally, for example perlingually, buccally, intravenously, nasally, rectally or inhalatively.

For human use, in the case of oral administration, it is good practice to administer doses of from 0.001 to 50 mg/kg, preferably of 0.01 mg/kg-20 mg/kg. In the case of parenteral administration, such as, for example, via mucous membranes nasally, buccally or inhalatively, it is good practice to use doses of 0.001 mg/kg-0.5 mg/kg.

In spite of this, if appropriate it may be necessary to depart from the amounts mentioned, namely depending on the body weight or the type of administration route, on the individual response towards the medicament, the manner of its formulation and the time or interval at which administration takes place. Thus, in some cases it may be adequate to manage with less than the abovementioned minimum amounts, while in other cases the upper limit mentioned has to be exceeded. In the case of the administration of relatively large amounts, it may be advisable to divide these into several individual doses over the course of the day.

The compounds according to the invention are also suitable for use in veterinary medicine. For use in veterinary medicine, the compounds or their non-toxic salts can be administered in a suitable formulation in accordance with general veterinary practice. Depending on the kind of animal to be treated, the veterinary surgeon can determine the nature of use and the dosage.

Starting Materials

### EXAMPLE 1A

### 2-Butyrylaminopropionic acid

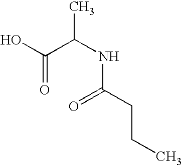

22.27 g (250 mmol) of D, L-alanine and 55.66 g (550 mmol) of triethylamine are dissolved in 250 ml of dichloromethane, and the solution is cooled to 0° C. 59.75 g (550 mmol) of trimethylsilyl chloride are added dropwise, and the solution is stirred at room temperature for 1 hour and at 40° C. for one hour. After cooling to −10° C., 26.64 g (250 mmol) of butyryl chloride are added dropwise, and the resulting mixture is stirred at −10° C. for 2 hours and at room temperature for one hour.

With ice-cooling, 125 ml of water are added dropwise and the reaction mixture is stirred at room temperature for 15 minutes. The aqueous phase is evaporated to dryness, the residue is triturated with acetone and the mother liquor is filtered off with suction. The solvent is removed, and the residue is then chromatographed. The resulting product is dissolved in 3N aqueous sodium hydroxide solution and the resulting solution is evaporated to dryness. The residue is taken up in conc. HCl and again evaporated to dryness. The residue is stirred with acetone, the precipitated solid is filtered off and the solvent is removed under reduced pressure. This gives 28.2 g (71%) of a viscous oil which crystallizes after a while.

200 MHz ¹H-NMR (DMSO-d₆): 0.84, t, 3H, 1.22, d, 3H, 1.50 hex, 2H, 2.07, t, 2H; 4.20, quin., 1H, 8.09, d, 1H.

### EXAMPLE 2A

### 2-Ethoxybenzonitrile

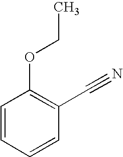

25 g (210 mmol) of 2-hydroxybenzonitrile, 87 g of potassium carbonate and 34.3 g (314.8 mmol) of ethyl bromide in 500 ml of acetone are refluxed overnight. The solid is filtered off, the solvent is removed under reduced pressure and the residue is distilled under reduced pressure. This gives 30.0 g (97%) of a colourless liquid.

200 MHz ¹H-NMR (DMSO-d₆): 1.48, t, 3H, 4.15, quart., 2H, 6.99, dt, 2H, 7.51, dt, 2H.

### EXAMPLE 3A

### 2-Ethoxybenzamidine hydrochloride

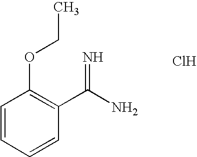

21.4 g (400 mmol) of ammonium chloride are suspended in 375 ml of toluene, and the suspension is cooled to 0° C. 200 ml of a 2M solution of trimethylaluminium in hexane are added dropwise, and the mixture is stirred at room temperature until the evolution of gas has ceased. 29.44 g (200 mmol) of 2-ethoxybenzonitrile are added, and the reaction mixture is then stirred at 80° C. (bath) overnight.

With ice-cooling, the cooled reaction mixture is added to a suspension of 100 g of silica gel and 950 ml of chloroform, and the mixture is stirred at room temperature for 30 minutes. The mixture is filtered off with suction and the residue is washed with the same amount of methanol. The mother liquor is concentrated and the resulting residue is stirred in a mixture of dichloromethane and methanol (9:1), the solid is filtered off with suction and the mother liquor is concentrated. This gives 30.4 g (76%) of a colourless solid.

200 MHz ¹H-NMR (DMSO-d₆): 1.36; t, 3H, 4.12, quart., 2H, 7.10, t, 1H, 7.21, d, 1H, 7.52, m, 2H, 9.30, s, broad, 4H.

### EXAMPLE 4A

### 2-(2-Ethoxyphenyl)-5-methyl-7-propyl-3H-imidazo[5,1-f]-1,2,4-triazin-4-one

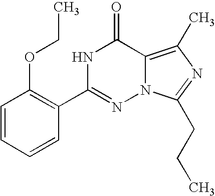

7.16 g (45 mmol) of 2-butyrylaminopropionic acid and 10.67 g of pyridine are dissolved in 45 ml of THF and, after addition of a spatula tip of DMAP, heated at reflux. 12.29 g (90 mmol) of ethyl oxalyl chloride are slowly added dropwise, and the reaction mixture is refluxed for 3 hours. The mixture is poured into ice-water and extracted three times with ethyl acetate, and the extracts are dried over sodium sulphate and concentrated. The residue is taken up in 15 ml of ethanol and refluxed with 2.15 g of sodium bicarbonate for 2.5 hours. The cooled solution is filtered.

With ice-cooling, 2.25 g (45 mmol) of hydrazine hydride are added dropwise to a solution of 9.03 g (45 mmol) of 2-ethoxybenzamidine hydrochloride in 45 ml of ethanol, and the resulting suspension is stirred at room temperature for 10 minutes. The ethanolic solution described above is added to this reaction mixture, and the mixture is stirred at a bath temperature of 70° C. for 4 hours. Following filtration, the solution is concentrated, the residue is partitioned between dichloromethane and water, the organic phase is dried over sodium sulphate and the solvent is removed under reduced pressure.

This residue is dissolved in 60 ml of 1,2-dichloroethane and, after addition of 7.5 ml of phosphorus oxychloride, refluxed for 2 hours. The mixture is diluted with dichloromethane and neutralized by addition of sodium bicarbonate solution and solid sodium bicarbonate. The organic phase is dried and the solvent is removed under reduced pressure. Chromatography with ethyl acetate and crystallization gives 4.00 g (28%) of a colourless solid, R_{f}=0.42 (dichloromethane/methanol=95:5).

200 MHz ¹H-NMR (CDCl₃): 1.02, t, 3H, 1.56, t, 3H, 1.89, hex, 2H, 2.67, s, 3H, 3.00, t, 2H, 4.26, quart., 2H, 7.05, m, 2H, 7.50, dt, 1H; 8.17, dd, 1H, 10.00, s, 1H.

### EXAMPLE 5A

### 4-Ethoxy-3-(5-methyl-4-oxo-7-propyl-3,4-dihydroimidazo[5,1-f]-1,2,4-triazin-2-yl) benzenesulphonyl chloride

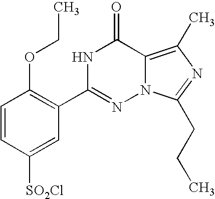

At 0° C., 2.00 g (6.4 mmol) of 2-(2-ethoxyphenyl)-5-methyl-7-propyl-3H-imidazo-[5,1-f]-1,2,4-triazin-4-one are added slowly to 3.83 ml of chlorosulphonic acid. The reaction mixture is stirred at room temperature overnight, poured into ice-water and extracted with dichloromethane. This gives 2.40 g (91%) of a colourless foam.

200 MHz ¹H-NMR (CDCl₃): 1.03, t, 3H, 1.61, t, 2H, 1.92, hex, 2H, 2.67, s, 3H, 3.10, t, 2H, 4.42, quart, 2H, 7.27, t, 1H; 8.20, dd, 1H; 8.67, d, 1H, 10.18 s, 1H.

### PREPARATION EXAMPLES

### Example 1

### 2-[2-Ethoxy-5-(1-piperazinylsulphonyl)phenyl]-5-methyl-7-propylimidazo-[5,1-f]-1,2,4-triazin-4(3H)-one

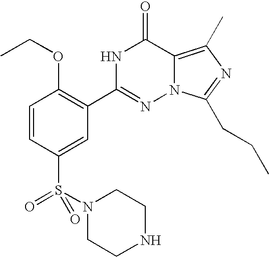

2.2 g (5.354 mmol) of the sulphonyl chloride from Example 5A are dissolved in 10 ml of dichloromethane and added dropwise to a solution of 4.61 g (53.54 mmol) of piperazine in 20 ml of dichloromethane. The mixture is stirred at RT for 10 min and the organic phase is washed with water, dried over sodium sulphate and concentrated. The product is recrystallized from ethyl acetate.

Yield: 1.83 g (74.2%)

M.p.: 256° C.

¹H-NMR (CD₃OD): δ=1.0 (t, 3H); 1.45 (t, 3H); 1.72 (sextett, 2H); 2.6 (s, 3H); 2.85-2.9 (m, 4H); 2.9-3.0 (m, 6H); 4.3 (q, 2H); 7.4 (d, 1H); 7.9 (dd, 1H); 8.0 (d, 1H).

### Example 2

### 2-{2-Ethoxy-5-[(4-ethyl-4-hydroxy-4λ⁵-piperazin-1-yl)sulphonyl]phenyl}-5-methyl-7-propylimidazo[5,1-f]-1,2,4-triazin-4(3H)-one

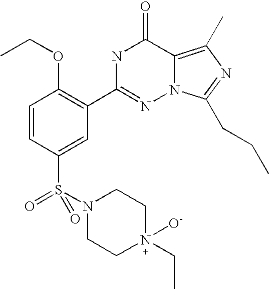

The preparation is carried out analogously to Example 1 using 0.69 g (1.67 mmol) of the sulphonyl chloride from Example 5A and 0.57 g (5 mmol) of ethylpiperazine. 0.5 g (1.023 mmol) of the resulting sulphonamide and 0.176 g (1.023 mmol) of 3-chloroperoxybenzoic acid are stirred in 5 ml of dichloromethane at RT for 1 h. The mixture is extracted 3× with saturated sodium carbonate solution, dried over sodium sulphate and concentrated. The residue is purified by chromatography on silica gel (mobile phase: dichloromethane/methanol 10: 1).

Yield: 0.13 g (25.2%)

M.p.: 224-225° C.

¹H-NMR (CD₃OD): δ=0.95 (t, 3H); 1.3 (t, 3H); 1.45 (t, 3H); 1.7 (sextett, 2H); 2.6 (s, 3H); 2.9-3.0 (m, 4H); 3.1-3.2 (m, 4H); 3.4-3.5 (m, 2H); 3.7 (d, 2H); 4.3 (q, 2H); 7.35 (d, 1H); 7.75 (dd, 1H); 8.05 (d, 1H)

### Example 3

### 4-Ethoxy-N-[2-(ethylamino)ethyl]-3-(5-methyl-4-oxo-7-propyl-3,4-dihydroimidazo[5,1-f]-1,2,4-triazin-2-yl)benzenesulphonamide

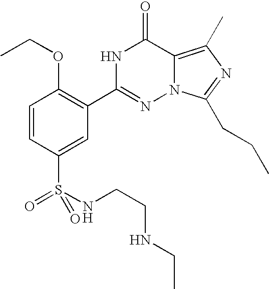

The preparation was carried out analogously to Example 1 using 2.2 g (5.35 mmol) of the sulphonyl chloride from Example 5A and 4.72 g (53.5 mmol) of ethylethylenediamine.

Yield: 1.4 g (56.5%)

M.p.: 148-150° C.

¹H-NMR (CD₃OD): δ=0.95 (t, 3H); 1.1 (t, 3H); 1.45 (t, 3H); 1.7 (sextett, 2H); 2.6 (s, 3H); 2.62 (q, 2H); 2.7 (t, 2H); 2.95 (t, 2H); 3.0 (t, 2H); 4.25 (q, 2H); 7.3 (d, 1H); 8.0 (dd, 1H); 8.1 (d, 1H)

### Example 4

### N-(2-aminoethyl)-4-ethoxy-3-(5-methyl-4-oxo-7-propyl-3,4-dihydroimidazo[5,1-f]-1,2,4-triazin-2-yl)benzenesulphonamide

1.0 g (2.434 mmol) of the sulphonyl chloride from Example 5A and 0.34 g (2.677 mmol) of glycine methyl ester hydrochloride, together with 0.57 g (5.598 mmol) of triethylamine, are stirred in 10 ml of dichloromethane at RT for 30 min. The mixture is extracted with dilute hydrochloric acid solution and then with saturated sodium chloride solution, and the organic phase is dried using sodium sulphate. The solvent is evaporated and the residue (0.96 g) is taken up in 20 ml of methanol and, after addition of 4.1 ml of 1 molar sodium hydroxide solution, stirred at RT for 3 h. The methanol is evaporated and the residue is treated with 10 ml of dilute HCl solution and extracted 2× with ethyl acetate. The organic phase is dried with sodium sulphate and then carefully concentrated, whereupon the product crystallizes out.

Yield: 0.307 g (33.3%)

¹H-NMR (DMSO): δ=0.9 (t, 3H); 1.3 (t, 3H); 1.7 (sextett, 2H); 2.45 (s, 3H); 2.85 (t, 2H); 3.6 (d, 2H); 4.2 (q, 2H); 7.35 (d, 1H); 7.85-7.95 (m, 2H); 8.1 (t, 1H)

### Example 6

### {[2-({[4-Ethoxy-3-(5-methyl-4-oxo-7-propyl-3,4-dihydroimidazo[5,1-f]-1,2,4-triazin-2-yl)phenyl]sulphonyl}amino)ethyl]amino}(oxo)acetic acid

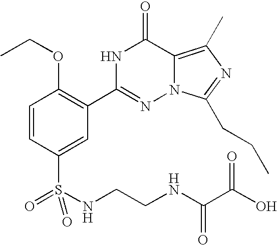

0.34 g (0.782 mmol) of the amine from Example 4 and 0.13 g (0.939 mmol) of ethyl oxalyl chloride, together with 0.2 g (1.956 mmol) of triethylamine, are stirred in 15 ml of dichloromethane at RT for 30 min. The mixture is concentrated and the residue is purified on silica gel (mobile phase: dichloromethane/methanol 50:1). This gives 0.18 g (43%) of the ethyl ester which is taken up in 5 ml of methanol. Following addition of 0.03 g (0.673 mmol) of sodium hydroxide in 2 ml of water, the mixture is stirred at RT for 30 min. The methanol is evaporated and the residue is treated with 5 ml of dilute HCl solution and extracted 2× with ethyl acetate. After drying over sodium sulphate, the solution is concentrated.

Yield: 0.023 g (13.5%)

¹H-NMR (CDCl₃/CD₃OD): δ=1.05 (t, 3H); 1.55 (t, 3H); 1.9 (sextett, 2H); 2.25 (s, 3H); 3.1-3.2 (m, 4H); 3.3-3.45 (m, 2H); 4.25-4.4 (q, 2H); 7.15 (d, 1H); 8.0 (dd, 1H); 8.3 (d, 1H)

### Example 7

### 2-{2-Ethoxy-5-[(3-oxo-1-piperazinyl)sulphonyl]phenyl}-5-methyl-7-propylimidazo[5,1-f]-1,2,4-triazin-4(3H)-one

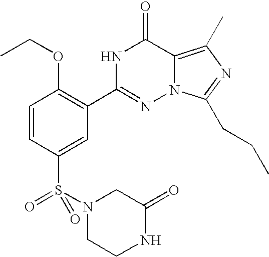

The preparation was carried out analogously to Example 1 using 0.66 g (1.606 mmol) of the sulphonyl chloride from Example 5A and 0.4 g (4.016 mmol) of 2-piperazinone.

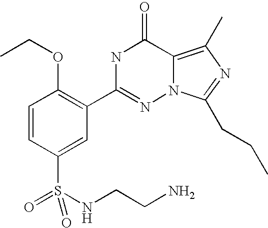

The preparation was carried out analogously to Example 1 using 2.2 g (5.35 mmol) of the sulphonyl chloride from Example 5A and 3.22 g (53.5 mmol) of ethylenediamine.

Yield: 1.13 g (48.6%)

M.p.: 226-228° C.

¹H-NMR (CD₃OD): δ=1.0 (t, 3H); 1.45 (t, 3H); 1.72 (sextett, 2H); 2.6 (s, 3H); 2.7 (t, 2H); 2.9-3.0 (m, 4H); 4.25 (q, 2H); 7.35 (d, 1H); 8.0 (dd, 1H); 8.1 (d, 1H)

### Example 5

### N-{[4-ethoxy-3-(5-methyl-4-oxo-7-propyl-3,4-dihydroimidazo[5,1-f]-1,2,4-triazin-2-yl)phenyl]sulphonyl}-glycine

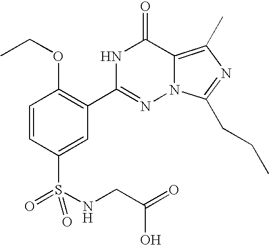

Yield: 0.613 g (80.4%)

¹H-NMR (CD₃OD): δ=1.0 (t, 3H); 1.45 (t, 3H); 1.8 (sextett, 2H); 2.6 (s, 3H); 2.95 (t, 2H); 3.3-3.4 (m, 4H); 3.7 (s, 2H); 4.3 (q, 2H); 7.4 (d, 1H); 8.0 (dd, 1H); 8.1 (d, 1H)

### Example 8

### 4-Ethoxy-3-(5-methyl-4-oxo-7-propyl-3,4-dihydroimidazo[5,1-f]-1,2,4-triazin-2-yl)benzenesulphonic acid

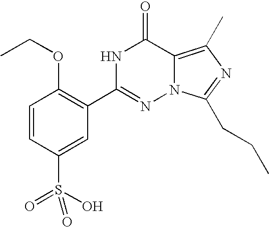

0.33 g (0.803 mmol) of the sulphonyl chloride from Example 5A are mixed with 10 ml of water and 5 ml of acetonitrile and stirred at room temperature for 18 hours. The resulting solution is then concentrated and the residue is dissolved in 60 ml of acetonitrile and filtered. The filtrate is concentrated again.

Yield: 0.28 g (88.7%)

¹H-NMR (CD₃OD): δ=0.95 (t, 3H); 1.45 (t; 3H); 1.7 (sextett; 2H); 2.6 (s, 3H); 2.7 (t, 2H); 4.25 (q, 2H); 7.35 (d, 1H); 8.0 (dd, 1H); 8.1 (d, 1H)

### Example 9

### 4-Ethoxy-3-(5-methyl-4-oxo-7-propyl-3,4-dihydroimidazo[5,1-f]-1,2,4-triazin-2-yl)benzenesulphonamide

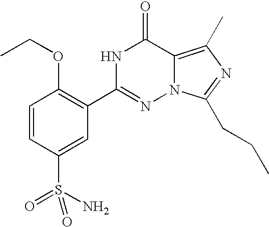

0.33 g (0.803 mmol) of the sulphonyl chloride from Example 5A are treated with 5 ml of 25% strength ammonia solution and stirred at room temperature for 2 hours. The solvent is then removed under reduced pressure. The residue is suspended in 10 ml of ice-water, filtered off, washed twice with in each case 10 ml of ice-water and dried in a vacuum desiccator.

Yield: 0.266 g (85.0%).

¹H-NMR (CD₃OD): δ=1.0 (t, 3H); 1.45 (t; 3H) 1.75 (sextett; 2H); 2.6 (s, 3H); 2.7 (t, 2H); 4.25 (q, 2H); 7.3 (d, 1H); 8.0 (dd, 1H); 8.1 (d, 1H)

1. Compounds of the general formula (I) 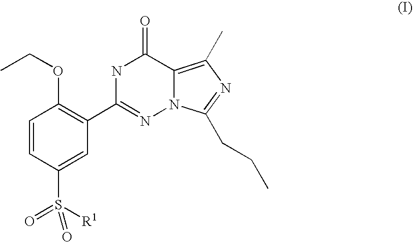
in which
R¹ represents 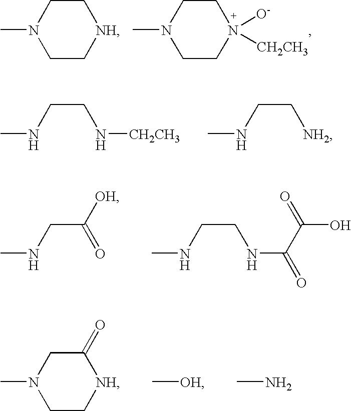
and their salts and hydrates.

2. (canceled)

3. Process for preparing compounds according to claim 1, characterized in that compounds of the formula (II) 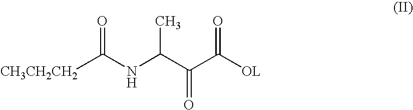
in which
L represents straight-chain or branched alkyl having up to 4 carbon atoms
are converted using the compound of the formula (III) 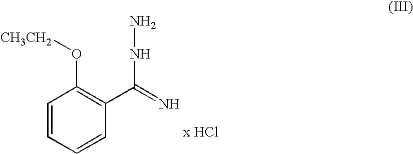
in a two-step reaction in the systems ethanol and phosphorus oxytrichloride/dichloroethane into the compound of the formula (IV) 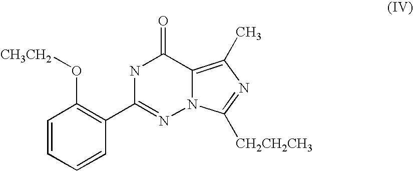
which, in a further step, is converted with chlorosulphonic acid into the compound of the formula (V) 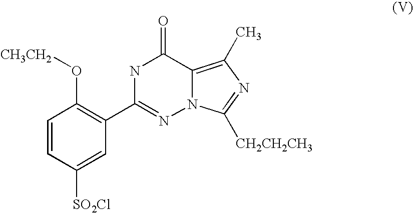
which is subsequently reacted with the corresponding amines in inert solvents to give the sulphonamides or converted into the free sulphonic acid.

4. A pharmaceutical composition comprising at least one compound according to claim 1 and pharmaceutically acceptable formulating agents.

5. A method for treating cardiovascular and cerebrovascular disorders and/or disorders of the urogenital tract comprising administering to a subject in need thereof an effective amount of a compound of claim 1.

6. The method of claim 5 wherein the disorder of the urogenital tract is erectile dysfunction.

7. (canceled)

8. (canceled)
